(19) United States
(12) Patent Application Publication
(10) Pub. No.: US 2017/0189201 A1
(43) Pub. Date: Jul. 6, 2017

(54) Cervical Intervertebral Cage with Increased Bone Contact Area and Improved Stability

(71) Applicant: DONGGUK UNIVERSITY GYEONGJU CAMPUS INDUSTRY-ACADEMY COOPERATION FOUNDATION, Gyeonsangbuk-do (KR)

(72) Inventor: Jae Jun Yang, Gyeonggi-do (KR)

(21) Appl. No.: 15/393,547

(22) Filed: Dec. 29, 2016

(30) Foreign Application Priority Data
Dec. 31, 2015 (KR) 10-2015-0191096

(51) Int. Cl. A61F 2/44

(52) U.S. Cl. CPC A61F 2/447; A61F 2310/00161; A61F 2310/00359; A61F 2310/00179; A61F 2002/30065

(57) ABSTRACT

The present invention relates to a cervical intervertebral cage with improved stability. Unique structures of the cervical intervertebral cage are wing-shaped protrusions. The protrusions come into contact with the bilateral uncinate processes of the cervical spine to improve stability and bone contact of the cage so that complications after ACDF using cervical cages such as cage subsidence and nonunion can be minimized.

Therefore, the cervical intervertebral cage of the present invention might be effectively used for surgical treatment of the cervical spine.

### CROSS-REFERENCE TO RELATED APPLICATION

This application claims priority to and the benefit of Korean Patent Application No. 10-2015-0191096, filed on Dec. 31, 2015, the disclosure of which is incorporated herein by reference in its entirety.

### BACKGROUND

1. Field of the Invention

The present invention relates to a cervical intervertebral cage, and more particularly, to a cervical intervertebral cage with an increased bone contact area and improved stability.

2. Discussion of Related Art

In recent years, anterior cervical discectomy and fusion (ACDF) has been commonly used for the purpose of surgical treatment of degenerative cervical disorders or cervical spinal traumas. Typically, autogenous bone grafting has been used for intervertebral fusion, but a cervical intervertebral cage has been widely used so far due to problems of autogenous bone itself such as low initial stability, collapse and prolapse of grafted bone, and complications by autogenous bone harvesting such as pain or infection in the donor sites. Although good clinical results for ACDF using cervical cages have been reported in the literature, cage subsidence and nonunion have been pointed out as the major complications of ACDF using cervical cages and additional fixation using metal plates or screws with the cages has been recently used in an effort to minimize the complications. In a perspective of designing a cervical cage to minimize the complications of cage subsidence and nonunion, it would be critical to maximize a bone contact and stability of a cervical cage. However, materialization of such cage designs might be challenging in considering unique anatomical characteristics of the cervical spine. A vertebral body of the cervical spine has endplates formed with thick cortical bone in the upper and lower margins in the axial plane. The uncinate processes, unique three dimensional bony structures of the cervical spine, are located at the posterolateral side of an upper endplate in a shape of a pair of symmetrical upward bony protrusion. The uncinate processes have increasing width from the front to the back and, therefore, a contact surface with a cervical cage in the upper endplate is inevitably limited. Considering the above anatomical characteristics of the cervical spine, a trapezoidal cage design with a wider front and a narrower back is widely used. However, since the uncinated processes have various shapes and sizes depending on segments of the cervical spine and individuals, it is difficult to secure a sufficient cage-bone contact surface using the trapezoidal cage design alone. Further, most spine surgeons pay attention to selection of proper cage heights neglecting cage widths for convenience of surgery even though cages with diverse widths and anteroposterior lengths are available on the market. Therefore, currently available cages have limitation in securing maximum cage-bone contact and stability of cages, and there has been no breakthrough concept of cage designs to overcome the limitation (Korean Registered Patent No. 10-1225006).

### SUMMARY OF THE INVENTION

The present invention is designed to solve the problems of the prior art, and therefore it is an object of the present invention to provide a cervical intervertebral cage with improved bone contact and stability.

However, the technical object of the present invention is not limited to that mentioned above, and other unmentioned objects will be clearly understood by those of ordinary skill in the art by descriptions below.

In order to fulfill the objective of the present intervention, there is provided a cervical intervertebral cage which includes:

A body 100 inserted between vertebral bodies of the cervical spine and having a through hole 110 formed in the center thereof; and

protrusions 200 symmetrically formed in both sides of lateral walls of the body 100.

Preferably, the lateral and bottom surfaces of each of the protrusions 200 may come into contact with an uncinated process to widen a bone contact area and fix the cervical intervertebral cage, thereby improving stability.

Preferably, the protrusions 200 may be formed in a wing shape.

Preferably, the lateral and bottom surface of each of the protrusions 200 may has a pattern of upside down stairs.

Preferably, the cervical intervertebral cage may be formed of polyetheretherketone (PEEK), carbon fibers, ceramics, a titamum alloy material, or allogenic bone.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a perspective view of a cervical intervertebral cage 10 according to one exemplary embodiment of the present invention;

FIG. 2 is a top view of the cervical intervertebral cage 10 according to one exemplary embodiment of the present invention;

FIG. 3 is a front view of the cervical intervertebral cage 10 according to one exemplary embodiment of the present invention; and

FIG. 4 is a diagram showing the cervical intervertebral cage 10 according to one exemplary embodiment of the present invention inserted between a superior cervical vertebral body and an inferior cervical vertebral body.

### DETAILED DESCRIPTION OF EXEMPLARY EMBODIMENTS

Below, a description will be given of preferred embodiments of the present invention in conjunction with the accompanying drawings. Throughout the accompanying drawings, the same reference numerals are used to designate the same or similar components. In the description of the present invention, when it is determined that the detailed description of the related art would obscure the gist of the present invention, the description thereof will be omitted. Also, in the drawings, the shapes and dimensions may be exaggerated for clarity, and the same reference numerals will be used throughout to designate the same or like components.

Unless the context clearly demands otherwise, throughout the description and the claims, the term “connected to” is intended to encompass the situation of “'connected indirectly to' through an element” as well as “connected directly to”. Unless the context clearly requires otherwise, throughout the description and the claims, the words “comprise”, “comprising” and the like are to be construed in an inclusive sense as opposed to an exclusive or exhaustive sense, that is to say, in the sense of “including, but not limited to”.

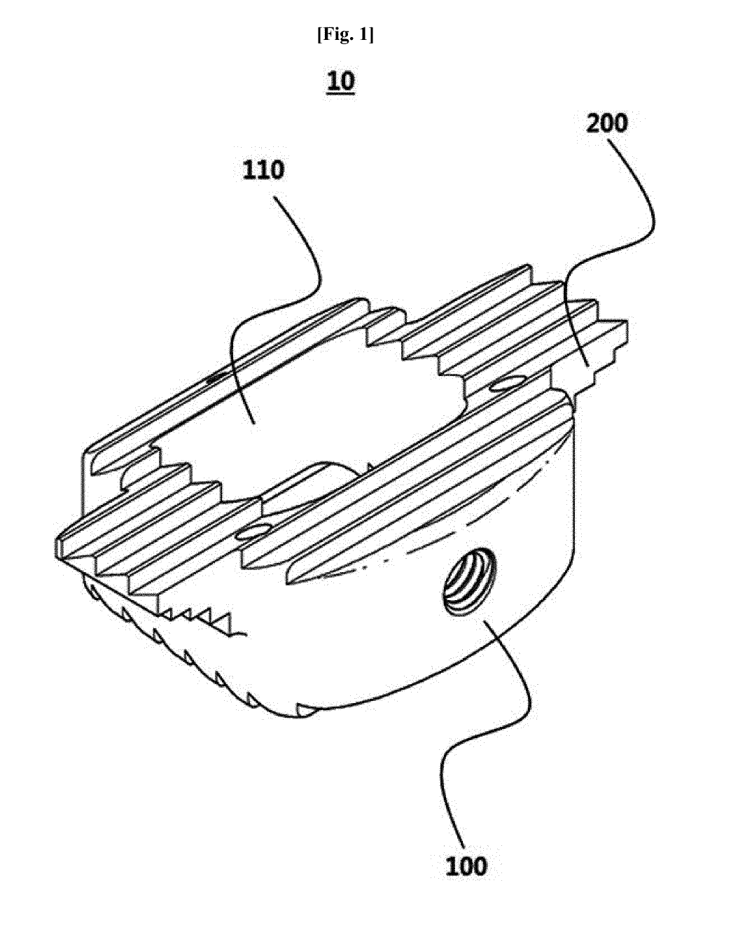

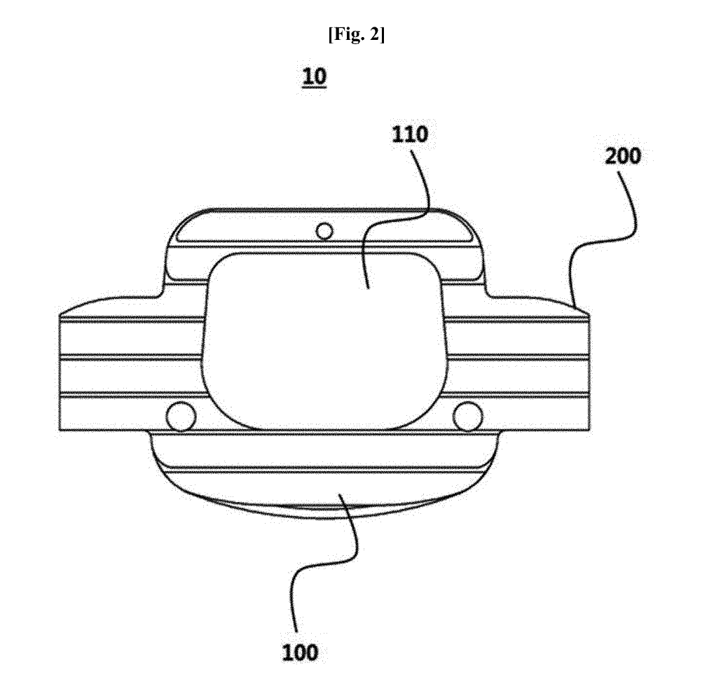

FIGS. 1 to 3 are a perspective view, a top view, and a front view of a cervical intervertebral cage 10 according to one exemplary embodiment of the present invention. As shown in FIGS. 1 to 3, the cervical intervertebral cage 10 according to one exemplary embodiment of the present invention may be configured to include a body 100 and (lateral) protrusions 200.

The cervical intervertebral cage 10 according to one exemplary embodiment of the present invention is an artificial structure that is inserted to and fixed in an intervertebral disc space after removal of the disc to form a bone fusion between superior and inferior cervical vertebral bodies and restore and maintain cervical intervertebral distance. Considering 3 dimensional characteristics of the endplates and uncinated processes of the cervical spine, the cervical intervertebral cage 10 was designed to contain wing-shaped lateral protrusions serving as crossbeams traversing between bilateral uncinated processes disposed at both sides of a cervical vertebra. It is expected that the cervical intervertebral cage 10 facilitates dispersion of load transfer into a wide range of the endplate including the uncinate processes so that stability of the cage might be improved and complications after ACDF using cages such as cage subsidence and nonunion might be minimized.

Hereinafter, the respective components constituting the cervical intervertebral cage 10 according to one exemplary embodiment of the present invention will be described in further detail.

The body 100 is configured to form a main frame of the cervical intervertebral cage 10 which is inserted to and fixed in an intervertebral disc space after removal of the disc. As shown in FIG. 1, the body 100 may be generally formed in a shape corresponding to the bony structure of the endplates and intervertebral disc space. Also, a through hole configured to pass through the body 100 in a vertical direction is formed at the center of the body 100. In this case, the through hole serves to connect superior and inferior cervical vertebral bodies, which are disposed on and under the removed intervertebral disc, so as to promote bone fusion between the superior cervical vertebral body and the inferior cervical vertebral body.

The height of the uncinate processes in the cervical vertebra differs by 1 mm or less in average in all segments other than the front portions of 6^{th} and 7^{th} cervical spine, compared to the height of the intervertebral disc in normal populations. This anatomical data indicates that the cervical intervertebral cage 10 with lateral protrusions contacting with the superomedial margins of the bilateral uncinate processes may provide restoration of proper height of the intervertebral space. Also, the width between the superomdial margins of bilateral uncinate processes has a relatively constant measured value with difference less than 2 mm from the front to the back in normal populations, compared to the width of the endplates tapering to the back. Therefore, considering such anatomical characteristics, the cage 10 with protrusions contacting with the uncinate processes can maximize the stability and bone contact more easily than conventional cages.

The protrusions 200 are configured to increase a contact area between the cage and the vertebral body, particularly a contact area between the cage and the inferior cervical vertebral body. As shown in FIG. 1, the protrusions 200 may be symmetrically formed in both sides of lateral walls of the body 100, preferably in a wing shape to effectively achieve desired functions.

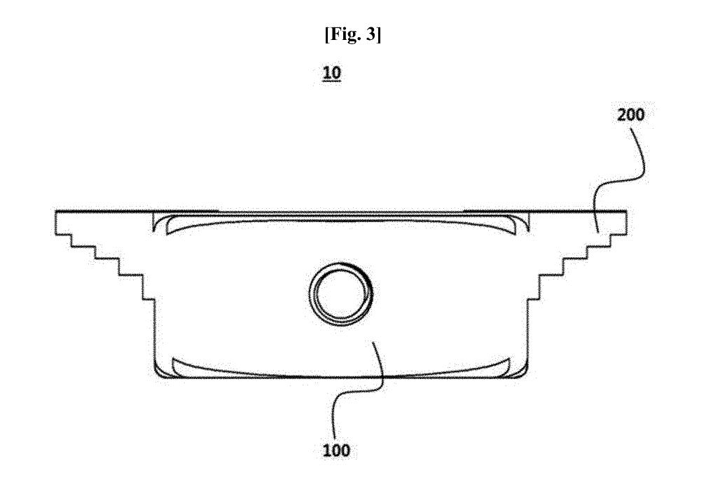

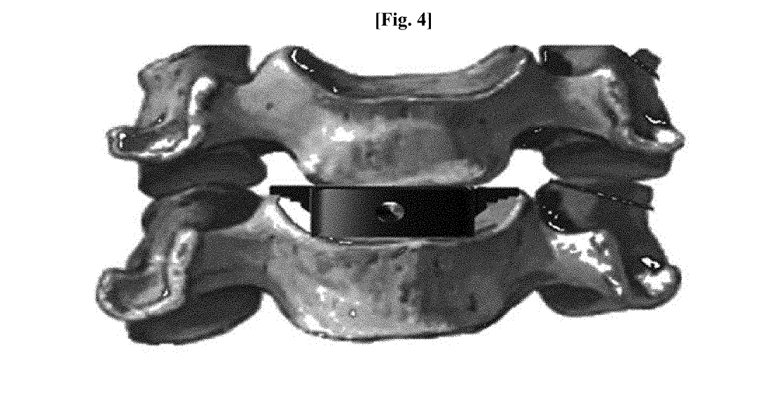

As shown in FIGS. 3 and 4, the protrusions 200 may come into contact with the uncinate processes in the inferior cervical vertebral body to enhance bone contact and fixation of the cervical intervertebral cage. Meanwhile, as shown in FIG. 3, the lateral and bottom surface of each of the protrusions 200 may have a pattern of upside down stairs so as to prevent slippage on a contact surface of the uncinate process and facilitate cutting of the protrusions for proper size, but the present invention is not limited thereto.

The cervical intervertebral cage 10 according to one exemplary embodiment of the present invention may be manufactured from a corrosion-resistant metal, ceramics, or allogenic bone, and is preferably manufactured from polyetheretherketone (PEEK), carbon fibers, ceramics, a titanium alloy material, or allogenic bone.

FIG. 4 is a diagram showing the cervical intervertebral cage 10 according to one exemplary embodiment of the present invention inserted in a cervical intervertebral disc space between superior and inferior cervical vertebral bodies. As shown in FIG. 4, the lateral and bottom surfaces of the protrusions 200 are fixed in between the bilateral uncinate processes to improve stability of the cage and disperse load transfer into wide areas of the inferior cervical vertebral body so that complications after ACDF using cervical cages such as cage subsidence and nonunion can be minimized.

It will be apparent to those skilled in the art that various modifications can be made to the above-described exemplary embodiments of the present invention without departing from the scope of the invention. Thus, it is intended that the present invention covers all such modifications provided they come within the scope of the appended claims and their equivalents.

What is claimed is:

1. A cervical intervertebral cage comprising:
a body (100) inserted between vertebral bodies of the cervical spine and having a through hole (110) formed in the center thereof; and
protrusions (200) symmetrically formed in both sides of lateral walls of the body (100).

2. The cervical intervertebral cage of claim 1, wherein lateral and bottom surfaces of each of the protrusions (200) come into contact with an uncinated process to widen a bone contact area and fix the cervical intervertebral cage, thereby improving stability.

3. The cervical intervertebral cage of claim 1, wherein the protrusions (200) are formed in a wing shape.

4. The cervical intervertebral cage of claim 1, wherein the lateral and bottom surface of each of the protrusions (200) has a pattern of upside down stairs.

5. The cervical intervertebral cage of claim 1, wherein the cervical intervertebral cage is formed of polyetheretherketone (PEEK), carbon fibers, ceramics, a titamum alloy material, or allogenic bone.
